(19) United States
(12) Patent Application Publication
(10) Pub. No.: US 2026/0080995 A1
(43) Pub. Date: Mar. 19, 2026

(54) PREDICTIVE MODELING OF THERAPEUTIC AGENT RESPONSE BASED ON A BODY COMPOSITION ANATOMICAL SEGMENTATION OF A CT SCAN

(71) Applicant: ONC.AI, INC., San Carlos, CA (US)

(72) Inventor: Petr Jordan, Emerald Hills, CA (US)

(21) Appl. No.: 18/885,154

(22) Filed: Sep. 13, 2024

(51) Int. Cl. G16H 20/10; G16H 10/40; G16H 10/60; G16H 30/40; G16H 50/70

(52) U.S. Cl. CPC G16H 20/10; G16H 10/40; G16H 10/60; G16H 30/40; G16H 50/70

(57) ABSTRACT

A system and method of predictive modeling of therapeutic agent response based on body composition anatomical segmentation of a computer tomography (CT) scan.

The method includes acquiring a single CT scan of one or more regions of a patient. The method includes segmenting the single CT scan to generate a volumetric segmentation (VS) mask indicative of a body composition anatomical segmentation of the patient. The method includes providing the VS mask to one or more predictive models trained to predict therapeutic agent responses based on the VS mask. The method includes generating, by a processing device, a predicted treatment response score to a treatment for the patient based on the VS mask and the one or more predictive models.

### TECHNICAL FIELD

The present disclosure relates to predicting therapeutic agent response in specific patients using deep learning analysis, and in particular to systems and methods of predictive modeling of therapeutic agent response based on a body composition anatomical segmentation of a computer tomography (CT) scan.

### BRIEF DESCRIPTION OF THE DRAWINGS

The present disclosure will be understood more fully from the detailed description given below and from the accompanying drawings of various implementations of the disclosure.

FIG. 1 is a diagram showing an exemplary embodiment of machine learning system for use with various embodiments of the present disclosure;

FIG. 2 depicts a flow diagram of a method of predicting immunotherapy treatment using deep learning analysis, in accordance with embodiments of the disclosure;

FIG. 3 is a diagram showing the patient imaging collection and treatment timeline, according to some embodiments;

FIG. 4A is an illustration of an example of a pre-treatment CT image (e.g., a 3D CT scan corresponding to a pre-baseline scan 304s or baseline scan 308s) of a target, in accordance with embodiments of the disclosure;

FIG. 4B is an illustration of an example of a follow-up image 350 of a target, in accordance with embodiments of the disclosure;

FIG. 5 is a diagram depicting an example environment 500 for generating volumetric segmentation (VS) masks based on a 3D CT scan used to train deep learning models for predicting therapeutic agent responses in specific patients, according to some embodiments;

FIG. 6 is a diagram depicting a VS mask that represents the anatomical structures of a patient from an axial view 602, a coronal view 604, and a sagittal view 606, according to some embodiments;

FIG. 7 is a diagram depicting a VS mask that represents the body composition anatomical segmentation of a patient using Skeletal Muscle Area (SMA) and Skeletal Muscle Density (SMD) from an axial view 702, a coronal view 704, and a sagittal view 706, according to some embodiments;

FIG. 8 is a diagram depicting a VS mask that represents the body composition anatomical segmentation of a patient using Visceral Fat Area (VFA) and Visceral Fat Density (VMD) from an axial view 802, a coronal view 804, and a sagittal view 806, according to some embodiments;

FIG. 9 is a diagram depicting a VS mask that represents the body composition anatomical segmentation of a patient using Subcutaneous Fat Area (SFA) and Subcutaneous Fat Density (SFD) from an axial view 902, a coronal view 904, and a sagittal view 906, according to some embodiments;

FIG. 10 depicts a flow diagram of a method for segmenting a CT scan into a volumetric segmentation mask indictive of a body composition anatomical segmentation of a patent for predictive modeling of therapeutic agent responses using deep learning analysis, according to some embodiments;

FIG. 11 depicts a flow diagram of a method for predicting therapeutic agent response for a specific patient using deep learning analysis of pre-treatment and intra-treatment serial 4D imaging of that specific patient, according to some embodiments; and

FIG. 12 illustrates a diagrammatic representation of a machine in the example form of a computer system within which a set of instructions, for causing the machine to perform any one or more of the methodologies discussed herein, may be executed.

### DETAILED DESCRIPTION

Embodiments of the present disclosure relate to the field of artificial intelligence, and in particular to systems and methods for generating volumetric segmentation (VS) masks based on a 3D CT scan (sometimes referred to herein as, CT scan) used to train deep learning models for predicting therapeutic agent responses in specific patients.

Predictive modeling of therapeutic agent responses can predict the likely outcome of treatment with a therapeutic with the aim of providing the physician with another tool to select the most appropriate therapeutic option for a given patient. That is, a predictive model may be built (e.g., trained) from a set of serial (e.g., longitudinal) features acquired prior to and during therapy, and used to predict an optimal therapy for the patient, such that adjustments may be made during the course of treatment, and/or to provide early insights and assessment of the therapeutic response. Examples of serial modeling features span many different data domains, e.g., levels of a given serum protein measured at different times, scans (e.g., computerized tomography (CT) scans) taken prior to and during therapy, a patient's cognitive performance status that is evaluated at each visit, etc. In some embodiments, a scan may include radiological images (e.g., CT scan, Magnetic Resonance Imaging (MRI), etc.).

The conventional techniques for predictive modeling are based on standardized body composition indices (e.g., Skeletal Muscle Index (SMI), Subcutaneous Fat Index (SFI), Visceral Fat Index (VFI)). The conventional techniques estimate the standardized body composition indices for a patient using segmentations of a single, 2D CT slice at the third lumbar spine vertebra (L3) of the patient.

However, the conventional technique's reliance on these known indices results in several drawbacks. For one, a predictive model of therapeutic agent response that is based on the conventional techniques that estimate a patient's standardized body composition have low and unreliable predictive accuracy. These conventional techniques also require for the patient's CT scan to include scanning information of the patient's L3 region. Most CT scans, however, do not include this information; thereby greatly limiting the range of uses of the conventional predictive modeling.

Furthermore, these conventional approaches for predicting therapeutic agent responses each use predictive models that are trained to make their predictions based on simple 3-dimentional (3D) CT scans that do not include any additional labeling information describing the structural components (e.g., body composition indices, anatomical structures) of the patient's body that was captured in the 3D CT scan. Therefore, the conventional predictive models are forced to make several assumptions when attempting to identify the different structures in the 3D CT scan and generate predictions based on the identified structures. Consequently, the conventional predictive models are at best inefficient and can also make grossly inaccurate predictions about a patient's response to treatment, which in turn, could add additional delay in a physician's attempt to identify the most effective treatment for the patient. Unfortunately, this additional delay could result in severe, if not fatal, consequences for the patient

Aspects of the present disclosure address the above-noted and other deficiencies by providing a preprocessing stage, prior to training the predictive model architecture, where the preprocessing automatically generates, using a segmentation algorithm, a VS mask that depicts the body composition anatomical segmentation of a 3D CT scan. The VS mask can be combined with the 3D CT scan to form (e.g., generate) a multi-channel data structure referred to as a 4D image (e.g., 3D CT scan overlaid on the VS mask). The 4D image can then be input into a deep learning model that is trained, using 4D images, to predict responses to a therapeutic agent based on the 4D image. Alternatively, the VS mask by itself (e.g., without the 4D image) can be input into a deep learning model that is trained, using only VS masks and/or VS masks and 4D images, to predict responses to a therapeutic agent based on the VS mask and/or the 4D image. That is, the deep learning model uses a patient's body composition to predict the patient's response to a therapeutic agent. Not by way of limitation, the responses may be used to select the optimal immunotherapy treatment plan for a particular patient with Non-Small Cell Lung Cancer (NSCLC). By training the predictive models using VS mask data and/or 4D image data instead of conventional 3D images (e.g., a CT scan), training efficiency and accuracy of predicted outcomes of the predictive models are significantly improved.

The terms “target,” “target lesion,” “target subject,” etc. may, for example, refer to a nodule, lesion, tumor, metastatic mass or an anatomical structure near (within some defined proximity to) a treatment area. In another embodiment, a target may be a bony structure or bone metastasis. In yet another embodiment a target may refer to soft tissue of a patient. A target may be any defined structure or area capable of being identified and tracked (including the entirety of the patient themselves) as described herein.

Furthermore, although a therapeutic agent (e.g., programmed cell death protein 1 (PD-1) agent, Cytotoxic T lymphocyte antigen 4 (CTLA-4) agent, etc.) is frequently referred to for convenience and brevity, the embodiments disclosed herein are similarly suitable for any other methods of treatment, including but not limited to other forms of immunotherapy, chemotherapy, and radiation therapy.

### 1. ML System for Predictive Modeling Therapeutic Agent Response

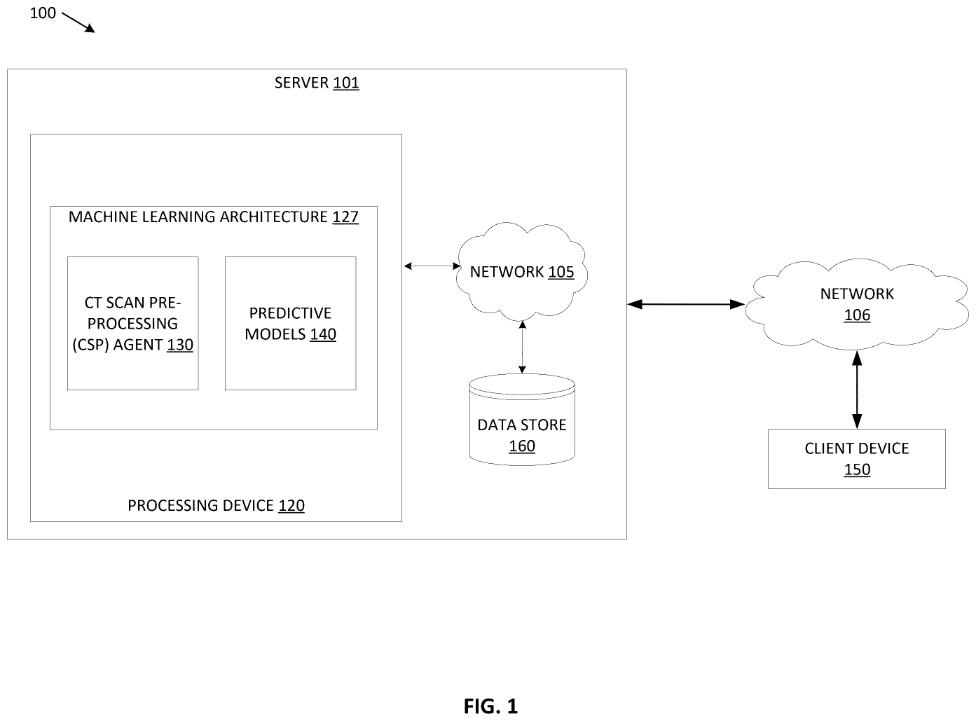

FIG. 1 is a diagram showing an exemplary embodiment of machine learning (ML) system 100 for use with various embodiments of the present disclosure. Although specific components are disclosed in machine learning system 100, it should be appreciated that such components are examples. That is, embodiments of the present disclosure are well suited to having various other components or variations of the components recited in machine learning system 100. It is appreciated that the components in machine learning system 100 may operate with other components than those presented, and that not all of the components of machine learning system 100 may be required to achieve the goals of machine learning system 100.

In one embodiment, the machine learning system 100 includes server 101, network 106, and client device 150. Server 101 may include various components, which may allow for using pre-treatment and/or intra-treatment serial imaging (available on server 101, client device 150, and/or data store 160) in predictive modeling and/or multi-modal predictive modeling of therapeutic agent response. Each component may perform different functions, operations, actions, processes, methods, etc., for a web application and/or may provide different services, functionalities, and/or resources for the web application. Server 101 may include machine learning architecture 127 of processing device 120 to perform operations related to using trained models to predict responses to one or more therapeutic agents using deep learning analysis of pre-treatment and/or intra-treatment serial imaging (e.g., images taken at different moments in time).

The machine learning architecture 127 includes a CT scan pre-processing (CSP) agent 130 and one or more predictive models 140. The CSP agent 130 is configured to pre-process (e.g., segment) a single 3D scan of one or more regions of a patient's body to generate additional information from the 3D scan. The additional information segments the structures of the patient's body that are captured in the CT scan. The one or more predictive models 140 can then use (in addition to the CT scan) the additional information to improve their capability and efficiency to predict the patient's response (e.g., therapeutic agent response) to treatment.

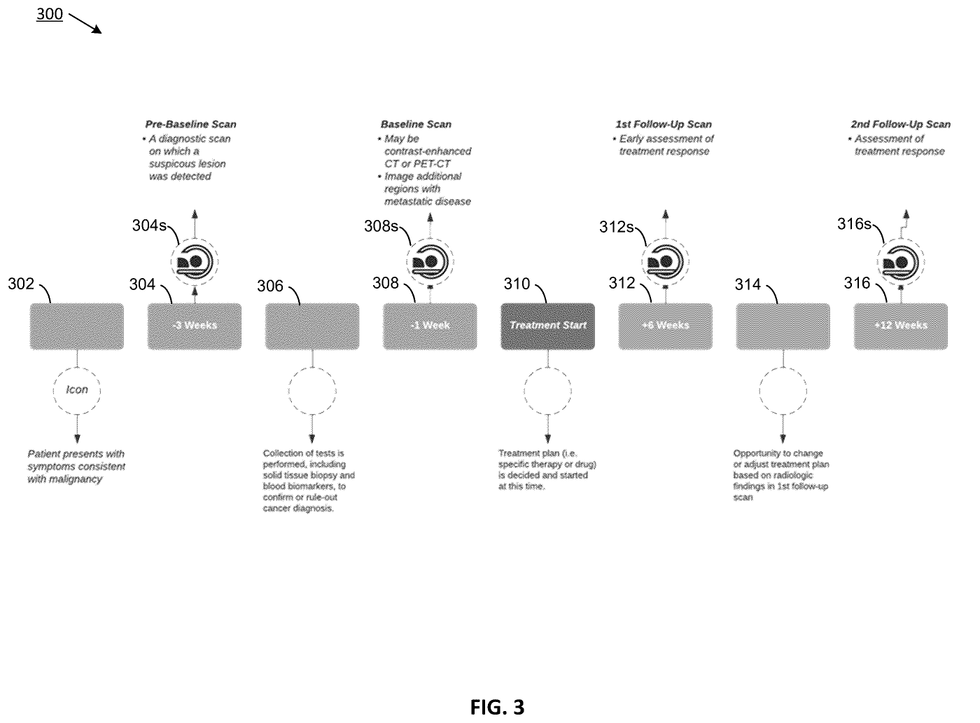

As further discussed herein, the CSP agent 130 is configured to identify or segment, based on the CT scan, various structures of the patient's body and generate one or more VS masks. A VS mask is a three dimensional (3D) depiction generated by segmenting body structures within a CT scan that can be displayed on a computing screen and in various views along axial, plane, and sagittal planes. Each of the VS masks include a plurality of labels (e.g., colors, text, symbols, and/or the like) indicating the different structures of the patient. The one or more VS masks are further discussed herein with respect to FIGS. 3-7.

The CSP agent 130 is configured to combine the one or more VS masks and the CT scan to generate a single 4D image that includes the different sets of labels. In some embodiments, the CSP agent combines the one or more VS masks and the CT scan by averaging the one or more VS masks and the CT scan to generate the single 4D image. The CSP agent 130 is configured to provide (e.g., input) the single 4D image to the one or more predictive models 140 for further processing. Alternatively, the CSP agent 130 may be configured to provide the VS mask by itself (e.g., without the 4D image) to the one or more predictive models 140 for further processing.

The one or more predictive models 140 are each configured to use the single 4D image to predict a therapeutic agent response and generate a predicted treatment response score that is indictive of the patient's response to treatment from the therapeutic agent. By providing a 4D image (e.g., a pre-segmented CT scan) to the one or more predictive models instead of only the CT scan (as is the case in conventional systems), the one or more predictive model 140 are able to make more informative and efficient predictions of the patient's response to treatment based on CT imaging. Advantageously, the predictions made by the one or more predictive models 140 are more efficient and accurate when derived from the analysis of 4D images instead of CT scans because a portion of the analysis is shifted from the one or more predictive models and placed onto the CSP agent 130, which is better equipped to perform a segmentation of the CT scan.

In one embodiment, processing device 120 may be one or more graphics processing units of one or more servers (e.g., including server 101). Additional details of machine learning architecture 127 are provided with respect to the remaining figures of the present disclosure. Server 101 may further include network 105 and data store 160.

The processing device 120 and the data store 160 are operatively coupled to each other (e.g., may be operatively coupled, communicatively coupled, may communicate data/messages with each other) via network 105. Network 105 may be a public network (e.g., the internet), a private network (e.g., a local area network (LAN) or wide area network (WAN)), or a combination thereof. In one embodiment, network 105 may include a wired or a wireless infrastructure, which may be provided by one or more wireless communications systems, such as a Wi-Fi hotspot connected with the network 105 and/or a wireless carrier system that can be implemented using various data processing equipment, communication towers (e.g., cell towers), etc. The network 105 may carry communications (e.g., data, message, packets, frames, etc.) between the various components of server 101. The data store 160 may be a persistent storage that can store data. A persistent storage may be a local storage unit or a remote storage unit. Persistent storage may be a magnetic storage unit, optical storage unit, solid state storage unit, electronic storage units (main memory), or similar storage unit. Persistent storage may also be a monolithic/single device or a distributed set of devices.

Each component may include hardware such as processing devices (e.g., processors, central processing units (CPUs), graphics processing units (GPUs), memory (e.g., random access memory (RAM), storage devices (e.g., hard-disk drive (HDD), solid-state drive (SSD), etc.), and other hardware devices (e.g., sound card, video card, etc.). The server 101 may comprise any suitable type of computing device or machine that has a programmable processor including, for example, server computers, desktop computers, laptop computers, tablet computers, smartphones, set-top boxes, etc. In some examples, the server 101 may comprise a single machine or may include multiple interconnected machines (e.g., multiple servers configured in a cluster). The server 101 may be implemented by a common entity/organization or may be implemented by different entities/organizations. For example, a server 101 may be operated by a first company/corporation and a second server (not pictured) may be operated by a second company/corporation. Each server may execute or include an operating system (OS), as discussed in more detail below. The OS of a server may manage the execution of other components (e.g., software, applications, etc.) and/or may manage access to the hardware (e.g., processors, memory, storage devices etc.) of the computing device.

As discussed herein, the server 101 may provide machine learning functionality to a client device (e.g., client device 150). In one embodiment, server 101 is operably connected to client device 150 via a network 106. Network 106 may be a public network (e.g., the internet), a private network (e.g., a local area network (LAN) or wide area network (WAN)), or a combination thereof. In one embodiment, network 106 may include a wired or a wireless infrastructure, which may be provided by one or more wireless communications systems, such as a Wi-Fi hotspot connected with the network 106 and/or a wireless carrier system that can be implemented using various data processing equipment, communication towers (e.g., cell towers), etc. The network 106 may carry communications (e.g., data, message, packets, frames, etc.) between the various components of server 101. Further implementation details of the operations performed by server 101 are described with respect to the remaining figures of the present disclosure.

Serial imaging in predictive modeling may be based on the observation that serial imaging captures changes in the appearance of lesions between pre-treatment and follow-up image, resulting from the therapeutic effect (or lack of effect) of the antineoplastic agent being administered. The embodiments of the present disclosure are centered around the observation that serial imaging performed prior to start of therapy can contain important insights about the aggressiveness (e.g., growth rate, volume, diameter) of each lesion. This is especially important in advanced stage disease with multiple tumor sites, where for example some tumor may be more stagnant, while other might exhibit aggressive growth rate. The tumor growth rate quantified from pre-treatment imaging is a powerful predictive feature that can be used in predictive models for antineoplastic agents (e.g., immunotherapy or targeted drug).

### 1.1 Overview of Using ML to Predict Immunotherapy Treatment Responses

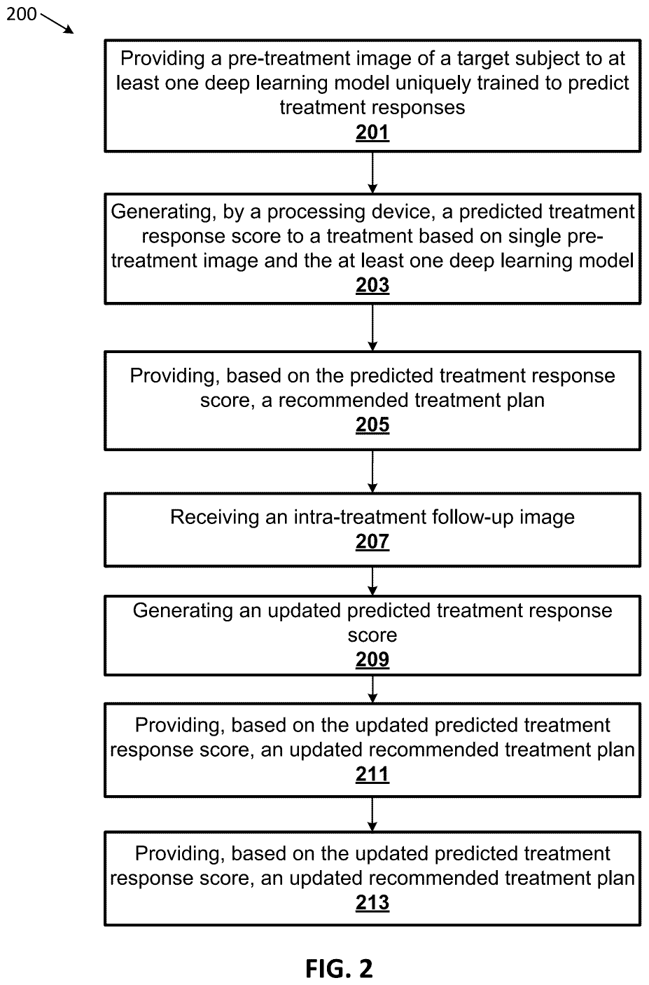

FIG. 2 depicts a flow diagram of a method of predicting immunotherapy treatment using deep learning analysis, in accordance with embodiments of the disclosure. Each of the methods described herein (including method 200) may be performed by processing logic that may include hardware (e.g., processing device, circuitry, dedicated logic, programmable logic, microcode, hardware of a device, etc.), software (e.g., instructions run or executed on a processing device), or a combination thereof. In some embodiments, the methods may be performed by processing logic (e.g., processing device 120) of the machine learning architecture 127 of FIG. 1.

As shown in FIG. 2, the method 200 includes the block 201 of providing a pre-treatment image of a target subject, optionally including lesion annotations or seed points, to at least one deep learning model uniquely trained to predict treatment responses (e.g., immunotherapy treatment) based on a single lesion or multiple lesions. In some embodiments, other types of machine learning models may be used instead of or in conjunction with the at least one deep learning model. In some embodiments, a large set of predefined imaging and clinical features is generated, followed by a feature selection algorithm (e.g., minimum redundancy maximum relevance (MRMR) or least absolute shrinkage and selection operator (LASSO)), and fitted using machine learning methods (e.g., gradient boosted decision trees, random decision forests, or support vector machines) to produce a predictive model. The optional lesion annotations or seed points provided to block 201 may be generated manually by the clinical user or automatically by an auto-segmentation and/or target detection method. An example of automatic auto-segmentation or target detection method is a convolutional neural network model. To predict treatment response of a single lesion, the predictive models 140 are trained using multiparametric optimization techniques, such as stochastic gradient descent (SGD), RMSprop, or adaptive momentum (Adam) algorithms, to maximize the agreement between model-predicted lesion response and lesion response determined by a human expert (e.g., radiologist).

A lesion response may include, for example, numerical assessment (e.g., change in lesion volume, change in one or more primary dimensions of the lesion, change in image intensity within the lesions), tumor growth rate (TGR), or categorical assessment (e.g., responding lesion, stable lesion, progressing lesion, new lesion).

Predicting treatment response at patient level is performed by aggregating one or more lesion-level model predictions. In some embodiments, aggregation from lesion to patient level response prediction is performed by a set of rules and/or logical operations.

In some embodiments, a per-lesion response score may be calculated for multiple lesions in a single patient, followed by a mathematical operation, such as maximum score, minimum score, and/or mean score to transform the multiple per-lesion response predictions into a single, patient-level response prediction. In some embodiments, aggregation from lesion to patient level response prediction is performed by a second model, which takes predictions from one or more lesion-level models as an input and is trained specifically to perform patient-level response prediction. In some embodiments, to account for variable numbers of lesions (e.g., the model inputs), the inputs into the model may be the lesion-level prediction statistics (e.g., mean, median, standard deviation, etc.). In another embodiment, the model may be a recurrent neural network (RNN) model in which multiple lesion predictions are represented as an input sequence of variable length.

A predictive model or deep learning model (each sometimes referred to as patient-level model) may include, for example, an artificial neural network, random forest model, support vector machine, and logistic regression model. In some embodiments, a single machine learning model may be used that considers multiple lesions at once; thereby effectively removing the hierarchy of per-lesion and per-patient models. In some embodiments, the pre-treatment image may be a two-dimensional anatomical image, a three-dimensional anatomical image, or a four-dimensional anatomical image. In another embodiment, two or more treatment images of a variety of types may be used.

The treatment image may be taken at the time of diagnosis (e.g., prior to start of treatment) or after the start of treatment. The treatment image may be, but is not limited to, a computed tomography (CT) scan, a positron emission tomography (PET) scan, or a magnetic resonance imaging (MRI) scan. A predictive model (e.g., deep learning model) may include any suitable variety of machine learning models including, but not limited to, a convolutional neural network. In some embodiments, the models are trained using the same sets of training data, different hyper-parameters, and/or different optimization techniques. In some embodiments, the models are trained using different sets of training data and different techniques having different objectives, etc., the results of which may be aggregated in a variety of ways.

The deep learning models may utilize a variety of suitable training methods. For example, in some embodiments, the deep learning models use a population of training subjects and a plurality of images associated with each of a plurality of training subjects as training data. In some embodiments, the deep learning models use calculated subject-specific models as training data. In some embodiments, the deep learning models use a combination of the two methods described above.

In some embodiments, the treatment is a PD-[L]1 immune checkpoint inhibitor treatment. The PD-[L]1 immune checkpoint inhibitor treatment may be a PD-1-based treatment or a PD-L1-based treatment. In some embodiments, the treatment is a CTLA-4-immune checkpoint inhibitor treatment, or any other suitable treatment type (e.g., chemotherapy, targeted therapy, pharmaceutical-based therapy, radiotherapy, etc.).

The method 200 includes the block 203 of generating a predicted treatment response score (e.g., on a scale representing least likely to have a positive of negative effect to most likely to have a positive or negative effect) to an immunotherapy treatment based on the deep learning models. In some embodiments, the predicted treatment response score may be a numerical value. In some embodiments, processing logic generates the predicted treatment response score based on the single pre-treatment image and the at least one deep learning model. For example, in some embodiments, results from the different models may be combined (e.g., averaged, or combined in any other way) to generate a single response score. In some embodiments, one or more non-imaging features (e.g., genomic tests, electronic medical record information, PD-L1 immunohistochemistry assays, etc.) may be used to generate the predicted response score. In another embodiment, the one or more non-imaging features may be combined with one or more imaging features to generate the predicted response score. Non-imaging features may include, for example, lab values, patient demographics, and/or molecular tests.

In some embodiments, the predicted treatment response score includes a prediction of patient progression on a predefined pharmaceutical product. In some embodiments, the predicted treatment response score indicates a prediction of one or more immune-related adverse events associated with the immunotherapy treatment. In some embodiments, the predicted treatment response score may include a predicted likelihood (e.g., a confidence level) of a specific type of response and/or adverse event occurring. In some embodiments, the response score may also include an indication of pseudo-progression, which is characterized by short-term and temporary increase in tumor volume due to natural swelling and/or inflammation (e.g., in response to treatment), rather than progression of disease. In some embodiments, the response score may reflect the likelihood of hyper-progression, which is a serious condition associated with rapid clinical deterioration and in which progression of disease is accelerated during administration of therapy. In some embodiments, the response score may be formulated to indicate a probability of progression-free survival or overall survival of cancer patients in units of months or years.

The method 200 includes the block 205 of providing, based on the predicted treatment response, a recommended treatment plan. For example, based on the predicted treatment response, a recommended treatment plan may include an indication of whether a specific pharmaceutical product should be used, a dosage of such product, a timing associated with administering such a product, etc. In some embodiments, the indication may identify whether or not a patient is likely to respond to the specific pharmaceutical product. In some embodiments, the per-lesion immunotherapy and/or chemotherapy response predictions are used to generate a lesion-specific therapy plan to enhance the therapeutic effect in high-risk lesions by combining ongoing systemic therapy with localized therapy. Localized therapy may be any of the following: stereotactic ablative radiation therapy (SBRT), intensity modulated radiation therapy (IMRT), conformal radiation therapy (CRT), radiosurgery, surgical resection, thermal ablation, cryoablation, or high intensity focused ultrasound (HIFU) therapy. In some embodiments, the recommended treatment plan for a patient with a model-predicted high risk of progression may be to add chemotherapy or CTLA-4 immunotherapy in combination with PD-[L]1 immunotherapy to maximize treatment response likelihood. In some embodiments, the recommended treatment plan may be to discontinue one or all therapeutic methods to maximize patient's quality of life. In some embodiments, the processing logic may generate other outputs based on the predicted treatment response score instead of or in conjunction with a recommended treatment plan. For example, the processing logic may generate a report based on the predicted treatment response score.

The method 200 includes the block 207 of receiving an intra-treatment follow-up image.

The method 200 includes the block 209 of providing the intra-treatment follow-up image to the machine learning model.

The method 200 includes the block 211 of generating an updated predicted treatment response score.

The method 200 includes the block 213 of providing, based on the updated predicted treatment response score, an updated recommended treatment plan.

The processing device 120 may perform any number of suitable pre-and post-processing operations that may increase the accuracy, efficiency, and/or compatibility of the machine learning model in the context at hand. For example, with respect to preprocessing, traditional radiomics methods may be susceptible to variations in scanner hardware and imaging protocols. The following data preprocessing and data augmentation systems are designed to optimize model generalizability and to minimize model susceptibility to imaging hardware and protocol variations:
1. Selecting model size (e.g., parameter count) that achieves optimal balance between underfitting and overfitting available training data. A) MLops (e.g., machine learning and operations) framework and infrastructure allows for the monitoring of model key performance indicators (KPIs) and for continually adjusting model complexity and architecture as more data is acquired.
2. Maximizing training dataset diversity. A) Training data may be sourced from diverse institutions (e.g., academic, small community centers, and large payer/provider networks), reflecting varying clinical practice trends and diverse imaging hardware and radiology protocols (e.g., some community cancer centers use CT protocols with thicker 5 mm slices, while research institutions tend to use high-resolution, 1-2 mm, thin slice scans). B) Training data may be internally cataloged using a database system and ensured proper distribution of imaging hardware and protocols when training models.
3. Input data normalization. A) During model training and model inference, scans may be resampled to consistent resolution (e.g., this may be 1.0×1.0×1.0 mm voxel spacing). This significantly reduces model performance dependence on CT slice thickness. B) Image voxel intensities may be normalized by excluding intensity outliers (e.g., metal artifacts from fiducials, pacemakers, wires, etc.) and rescaling the intensities to a consistent range (e.g., intensity distribution with 0 mean and variance of 1). C) In cases where multiple reconstructions protocols are available for a given imaging session, reconstruction protocol most consistent with a “gold standard” protocol may be used.
4. Augmenting training data by generating synthetic training examples that simulate feasible scenarios not represented in available training data. A) Online augmentation strategy may be used, which means that new variations of training data are continually generated as long as the model is being trained. In practice, this means that the number of unique training examples is infinite and is only limited by time spent in the model training loop. Online augmentation loops perform model shifts, rotations, rescaling operations, deformations, and intensity perturbations to generate new, unique training cases. B) Physics-based principles may be used to generated noise and intensity variations to simulate differences between scanner hardware and scanning protocols.

Examples of physics-based methods include raytracing and Monte-Carlo photon simulations on existing clinical CT scans to generate variations of CT projection data, which can subsequently be used to reconstruct new CT scans with alternate imaging protocols and simulated artifacts. Examples of simulated artifacts include different primary beam energies, beam scatter and hardening characteristics, patient motion artifacts, imaging dose variations.
5. Model inputs using multiple resolutions and region-of-interest (ROI) sizes. A) The CNN model may prefer a subregion (ROI) of one or more CT scans as an input. ROIs of varying size and resolution may be used to create a redundant representation of the input CT image (or subregion) in the vicinity of the tumor location. By using multiple ROI sizes, the model can accommodate for tumors of different size and shape. For example, if only an ROI spanning 5×5×5 cm around the tumor was used, the model would likely not perform well on large tumors. Conversely, if a 50×50×50 cm ROI was used, the classifier would likely not perform well for smaller tumors that require high spatial resolution and fidelity. Combining ROI regions with small and large spatial dimensions in one model facilitates complementary learning of imaging features at the local context (e.g., tumor shape, texture, and intensity profile) and at the global context (e.g., location of the lesion within the body and with respect to other organs, lymph node involvement, patient's body mass composition and muscle reserve, overall health or vital organs, microcalcifications, etc.) and may ultimately results in more predictive and more robust treatment response and survival prediction models.

With respect to post-processing, a variety of techniques may be used to post-process individual model predictions to obtain the predictions accuracy and explainability required by clinical end users. Examples of post processing methods used may include, but are not limited to:
1. Model Ensembles: ensembling (or bagging) is a method for improving stability and overall performance of models. Rather than training one model for a given task, multiple variations of a model are trained (by perturbing training hyper parameters, weight initializations, model architecture, training set distribution, etc.). The multiple models are then used simultaneously by calculating a consensus among them (ensemble prediction). In one embodiment, an average or median prediction from multiple models is on average more accurate than a single prediction. Examples of ensembling operations to combine multiple model predictions can be simple averaging, median calculating, the STAPLE algorithm (Simultaneous Truth and Performance Level Estimation, Warfield et. al.), or a dedicated ensembling model, such as linear classifier, random forest, support vector machine, or a neural network.
2. Bottom-up model aggregation: In some clinical applications, the concept of training a classification model for predicting single lesion response to a therapeutic agent may be desirable. In some clinical scenarios, the clinical requirement is to predict treatment response at the patient level (e.g., Will this patient benefit from given therapy overall, considering that some lesions may respond while others will continue to progress?). In this scenario, the concept of model ensembles may also be applicable. In this application, however, each single-lesion model (or sub-ensemble of models) contributes to the overall patient-level prediction, which is estimated by ensembling individual lesion predictions. Combining the prediction of each model within the larger ensemble and incorporating other clinical factors, biomarkers, and/or imaging features, processing logic can make predictions of treatment response at the patient level, rather than lesion level.
3. Explainability: The response of a deep convolutional network model can be broken down into activations of dominant features to highlight which spatial, textural, and morphologic features most influenced the prediction. For example, the explanation may predict “high risk of lesion progression” due to: 1. lesion volume greater than 50 cc, 2. lesion location in the apex of the lung, 3. low textural heterogeneity at the core and the perimeter of the lesion, 4. presence of metastatic bone lesions. In a related embodiment, model response prediction or a prediction of immune-related adverse events may be explained and supported by the processing unit by presenting reference data and historical cases of patients with similar presentation and medical history profiles.

Incorporating of Temporal Information: In one embodiment, the treatment prediction model can be thought of as either a “single shot” prediction at baseline that determines the future course of treatment, or as a continually integrated process that incorporates imaging and electronic medical record (EMR) information along the course of the treatment, providing continuous decision support for the clinician. In one embodiment, a treatment response model is trained to predict patient's likelihood of disease progression, pseudo-progression, or hyper-progression using baseline and first intra-treatment follow-up scan. In this clinical scenario, the model prediction may be used to significantly reduce the timeline to make treatment decision or adjustment, such as moving patient to a different therapeutic agent, adding a secondary therapeutic agent, or discontinuing therapy. In the case of prediction models which incorporate multiple imaging time points, temporal data can be integrated in various ways (two imaging time points may be used for illustration purposes):
1. Approach #1: Calculating the difference in imaging features between scan #1 and scan #2, which are subsequently used to create a prediction model. In one embodiment, sets of imaging features may be calculated independently for scan #1 and scan #1. The feature weights or values calculated from scan #1 may be subtracted from the features or values calculated from scan #2. The difference or changes in the individual features may constitute a set of new “delta features” that corresponds to temporal variations in typical image features (e.g., change in shape, intensity, texture, etc. as a function of time).
2. Approach #2: Training a 4D CNN prediction model with input ROI shape being [Nx, Ny, Nz, 2], where Nx, Ny, Nz are the number of voxels along each axis and 2 corresponds to two (or more) imaging time points, each represented with a single 3D volume within the 4D input volume). This approach is similar to multi-modal CNN models. The most obvious being natural images in RGB format, where each color channel is represented separately. In some embodiments, each channel is used for representing one event in time.
3. Approach #3: Calculating the intensity difference between spatially registered scans #1 and #2 and subsequently training a 3D CNN prediction model (model input ROI shape being [Nx, Ny, Nz, 1], where Nx, Ny, Nz are the number of voxels along each axis and 1 corresponds to single intensity channel).
4. Approach #4: Training a model combining 3D CNN with RNN (recurrent neural network), where the RNN is used to model sequence of imaging inputs.

### 1.2 Response Assessment (Labels)

Once a therapeutic agent is started, some lesions might decrease in size, while some highly aggressive lesions might only decelerate in terms of growth rate. The latter (e.g., change in growth rate) may be described as the second derivative of tumor volume with respect to time and it has the potential to quantify drug effects better than the traditional change in absolute lesion diameter (e.g., the response evaluation criteria in solid tumors (RECIST) protocol. This concept can also be described as lesion kinetics, where one is concerned with measuring the acceleration vs. velocity of tumor growth. This concept can be applied to single lesion at a time or to measure an aggregate of all lesions within one patient. Furthermore, different endpoints (e.g., outcomes) can be modeled (e.g., predicted) with this approach, including those typically employed in cancer drug trials, such as the overall survival (OS), progression-free survival (PFS), overall response rate (ORR) or individual tumor kinetics (e.g., velocity, acceleration). The resulting models incorporating these novel features and assessment labels can be formulated as either classification or regression models depending on the nature of the prediction. The architecture of such models can range from simple rule-based models, decision trees, random forest, support vector machines, all the way to deep neural networks.

In one embodiment, a predictive model uses changes in features (sometimes referred to as, novel features) extracted from pre-treatment images of one or more target lesions and is trained to predict a response assessment label including, for example, RECIST or tumor volume change from baseline.

In another embodiment, the same model uses pre-treatment multi-modal features (e.g., change in blood lab values, change in urine lab values, and change in imaging features) extracted from the pre-treatment images.

In additional embodiments, the imaging and multi-modal models are trained to predict response to therapy quantified in terms of change in growth rate, which the inventors have discovered as a novel response assessment method.

### 2. Patient Imaging Collection and Treatment

FIG. 3 is a diagram showing the patient imaging collection and treatment timeline, according to some embodiments. The diagram includes several time points (e.g., 302, 304, 306, 308) that occur pre-treatment, a time point 310 indicating when treatment starts, and several time point (e.g., 312, 314, and 316) that occur post-treatment. Although each time point is shown in FIG. 3 as being spaced apart in time by a particular time unit (e.g., −3 weeks, −1 week, etc.), the time points may be spaced apart in any time units (e.g., +/−minutes, +/−days, +/−months, etc.).

The CSP agent 130 of the machine learning architecture 127 may use a CT scan that was acquired from any time point prior to treatment start 310 to generate the one or more VS masks and then combine the VS masks to generate the single 4D image. For example, the CSP agent 130 may use a CT scan corresponding to a pre-baseline scan 304s or baseline scan 308s to generate a single 4D image.

At time point 302, a patient presents with symptoms consistent with malignancy. At time point 304 (e.g., −3 weeks), the server 101 acquires a pre-baseline scan 304s, which is a diagnostic scan on which a suspicious lesion was detected. At time point 306, the server 101 acquires (e.g., retrieves, receives) a collection of patient test data on solid tissue, biopsy, and blood biomarkers to confirm or rule-out cancer diagnosis. At time point 308 (e.g., −1 week), the server 101 acquires a Baseline Scan 308s, where the Baseline Scan 308s may be contrast-enhanced CT or PET-CT, and/or may include additional regions (e.g., anatomical structure) with metastatic disease.

The server 101 then uses its CSP agent 130 to generate a first 4D image based on the pre-baseline scan 304s and a second 4D image based on Baseline Scan 308s.

At time point 310 (e.g., t=0), the server 101 decides on a treatment plan (e.g., a specific therapy or drug) based on the first 4D image derived from the Pre-Baseline Scan 304s, patient test data, and/or the second 4D image derived from the Baseline Scan 308s. The server 101 then starts the patient on the treatment plan.

At time point 312 (e.g., +6 weeks after treatment), the server 101 acquires a 1^{st} follow-up scan 312s, which is an early assessment of the patient's response to the treatment. At time point 314, the server 101 may decide to adjust (e.g., modify) the treatment plan based on radiologic findings in the 1^{st} follow-up scan 312s, or may decide that no adjustment to the treatment plan should be made. In some embodiments, a radiologic finding may include a tumor growth rate, a tumor volume change, a tumor diameter change, and/or a tumor shape change, etc. At time point 316, the server 101 acquires a 2^{nd} follow-up scan, which is an assessment of the patient's response to the applied treatment, and again use its CSP agent 130 to generate a third 4D image based on the 2^{nd} follow-up scan.

### 2.1 Using 4D Images to Improve Efficiency and Accuracy of Model Training

As discussed herein, the server 101 uses a collection of training cases (e.g., training data) to train the one or more predictive models 140 to generate a predicted treatment response score that is indictive of a patient's response to a treatment. To improve efficiency and accuracy of the model training, the server 101 may also train the model using several items of information, but at a minimum, one or more 4D images which were each derived by the CSP agent 130 of the machine learning architecture 127 from one or more CT scans. That is, the CSP agent 130 generates a 4D image by segmenting a CT scan (e.g., 3D image) into one or more VS masks and combining the CT scan and the one or more VS masks into the 4D image. The server 101 then uses a one or more of the 4D images to train the one or more predictive models 140 to generate a predicted treatment response score that is indictive of the patient's response to a treatment. The server 101 may implement the following method to train the one or more predictive models 140.

In operation 1, the server 101 creates a collection (e.g., one or more) of training cases, consisting of retrospective longitudinal patient records consisting of serial imaging data (e.g., 4D images), medication treatment history, and/or non-imaging clinical features, etc.

In operation 2, for each training case, the server 101 extracts (e.g., determine, identify) model features and outcome labels (sometimes referred to as, “ground truth”).

In operation 3, the server 101 may extract model features according to the following method:

In operation 3a, the server 101 identifies target lesion(s) on Baseline Scan 208s immediately prior to Treatment Start 210. In operation 3b, the server 101 identifies corresponding target lesions(s) on Pre-Baseline Scan 204s. In operation 3c, the server 101 calculates (e.g., determines, measures) imaging and non-imaging Baseline Features from a Baseline scan 208s. In operation 3d, the server 101 calculates imaging and non-imaging Pre-Baseline Features from Pre-Baseline Scan 204s. In operation 3e, the server 101 calculates a difference or change in imaging and non-imaging features between Pre-Baseline Scan 204s and Baseline Scan 208s. The server 101 may normalize the change in features between Pre-Baseline Scan 204s and Baseline Scan 208s by dividing the change in imaging and non-imaging features by the number of days between the Pre-Baseline Scan 204s and Baseline Scan 208s to produce a normalized change.

In operation 4, the server extracts outcome features according to the following method:

In operation 4a, the server 101 identifies target lesion(s) on Baseline Scan 208s immediately prior to Treatment Start 210.

In operation 4b, the server 101 identifies corresponding target lesions(s) on 1^{st} Follow-up Scan 212s or 2^{nd} Follow-up Scan 216s.

In operation 4c, the server 101 calculates per-lesion response labels for each target lesion. In some embodiments, each label may be one of the following: a categorical variable (e.g., progressive disease, stable disease, partial response, complete response), a scalar variable corresponding to change in diameter, a scalar variable corresponding to absolute change in volume, a scalar variable corresponding to relative (e.g., percent change) in volume, a scalar variable corresponding to growth rate (e.g., linear or exponential change in volume per unit of time).

In operation 4d, the server 101 calculates per-patient response labels using one of the following methods: (a) simple mean (or median) of all per-lesion labels, minimum (or maximum) of all per-lesion labels; (b) categorical variable representing the following states: Uniform response (all target lesions responding to therapy), Uniform progression (all target lesions not responding to therapy and growing), mixed response (some target lesion responding and some progressing); according to known response assessment protocols (e.g. RECIST 1.1, iRECIST, irRECIST, etc.). In some embodiments, other patient-level outcome labels may include overall survival (e.g., at 6 months, 1 year, 2 years, etc.), change in therapy, treatment discontinuation, and/or immune-related adverse event, etc.

In some embodiments, the method for calculating features and labels describes the first difference (e.g., velocity) using two time points. An extension of this framework can be constructed where the server 101 uses 3 or more time points to calculate and use second difference (e.g., acceleration) in features and labels.

In some embodiments, the server 101 performs the feature selection method (using known algorithms) to identify a smaller subset of features that most closely associates with the chosen outcome label.

In some embodiments, the server 101 uses an optimization algorithm (e.g., stochastic gradient descent, ADAM, etc.) to train model(s) that, across all training cases, maximize the agreement between outcome labels and model predictions generated from model and its inputs (e.g., features).

### 2.2 Model Inference Procedure

The server 101 may perform a model inference according to the following method:

In operation 1, the server 101 identifies target lesion(s) on Baseline Scan 208s immediately prior to Treatment Start 210. In operation 2, the server 101 identifies corresponding target lesions(s) on Pre-Baseline Scan 204s. In operation 3, the server 101 calculates imaging and non-imaging Baseline Features from Baseline scan 208s. In operation 4, the server 101 calculates imaging and non-imaging Pre-Baseline Features from Pre-Baseline scan 204s.

In operation 5, the server 101 calculate a change in imaging and non-imaging features between Pre-Baseline Scan 204s and Baseline Scan 208s. In some embodiments, a change in features between Pre-Baseline Scan 204s and Baseline Scan 208s are normalized by the number of days between the two scans. For example, the server 101 divides the change in imaging and non-imaging features by the number of days between the Pre-Baseline Scan 204s and Baseline Scan 208s to produce a normalized change.

In operation 6, the server 101 combines Baseline Features, Pre-Baseline Features, and the differences/changes in these features as inputs to lesion-level and patient-level treatment response models to predict treatment response at specific time point after Treatment Start (e.g., +6 weeks, +12 weeks, etc.). In some embodiments, lesion-level models predict treatment response (e.g., growth kinetics) of each individual target lesion. In some embodiments, patient-level model combines predicted growth kinetics of each individual target lesion to combined patient-level response (e.g., in accordance with the RECIST assessment criteria).

In some embodiments, the server 101 may use lesion-level and patient-level predictions to create a treatment plan recommendation.

In some embodiments, the server 101 may collect observed lesion-level and patient-level outcome labels for Online Learning model adaptation.

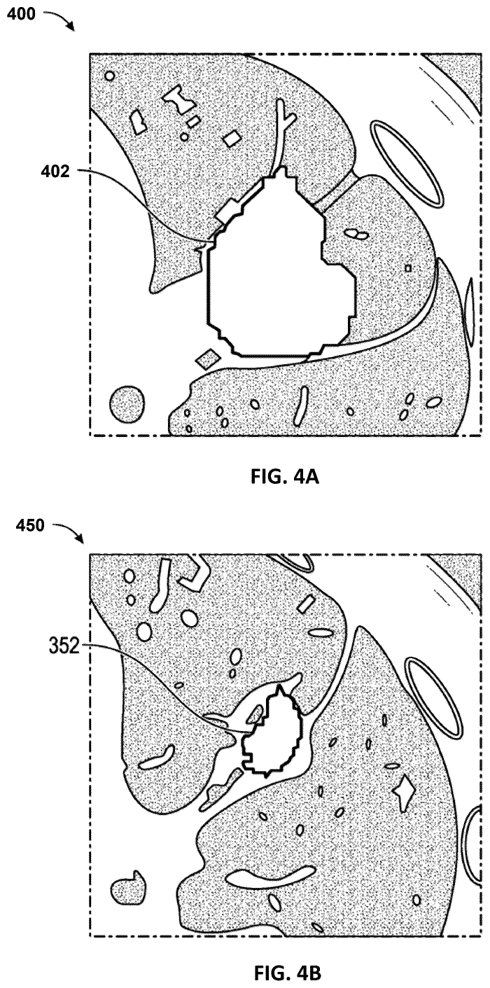

FIG. 4A is an illustration of an example of a pre-treatment CT image (e.g., a 3D CT scan corresponding to a pre-baseline scan 304s or baseline scan 308s) of a target, in accordance with embodiments of the disclosure. The pre-treatment image 300 may correspond to a lung lesion 402 of a patient at any time point (e.g., 302, 304, 306, 308) prior to administering or treating the patient according to a treatment plan. For example, the treatment plan may include treating the patient with immunotherapy. In some embodiments, a pre-treatment CT image may be, but is not limited to, a computed tomography (CT) scan, a positron emission tomography (PET) scan, or a magnetic resonance imaging (MRI) scan. In some embodiments, two or more treatment images of a variety of types (e.g., CT scan, PET scan, MRI scan) may be used.

FIG. 4B is an illustration of an example of a follow-up image 450 of a target, in accordance with embodiments of the disclosure. As previously described, embodiments of the disclosure may utilize one or more follow-up images, such as follow-up image 450, of the target that were captured after treatment. The follow-up image 450 includes lung lesion 452, which may correspond to lung lesion 402 after receiving treatment. In embodiments, the follow-up image 450 may be provided to the machine learning architecture 127 and may be used to determine whether the current treatment plan is effective and should continue, whether there is a more effective treatment option, and/or whether the treatment should be discontinued based on an analysis of the follow-up image 450 relative to pre-treatment image 400. In embodiments, the follow-up image 450 may correspond to a CT image. In some embodiments, the follow-up image 450 may correspond to a PET image. In an embodiment, the follow-up image 450 may correspond to an MRI image. In some embodiments, other types of follow-up images may be used.

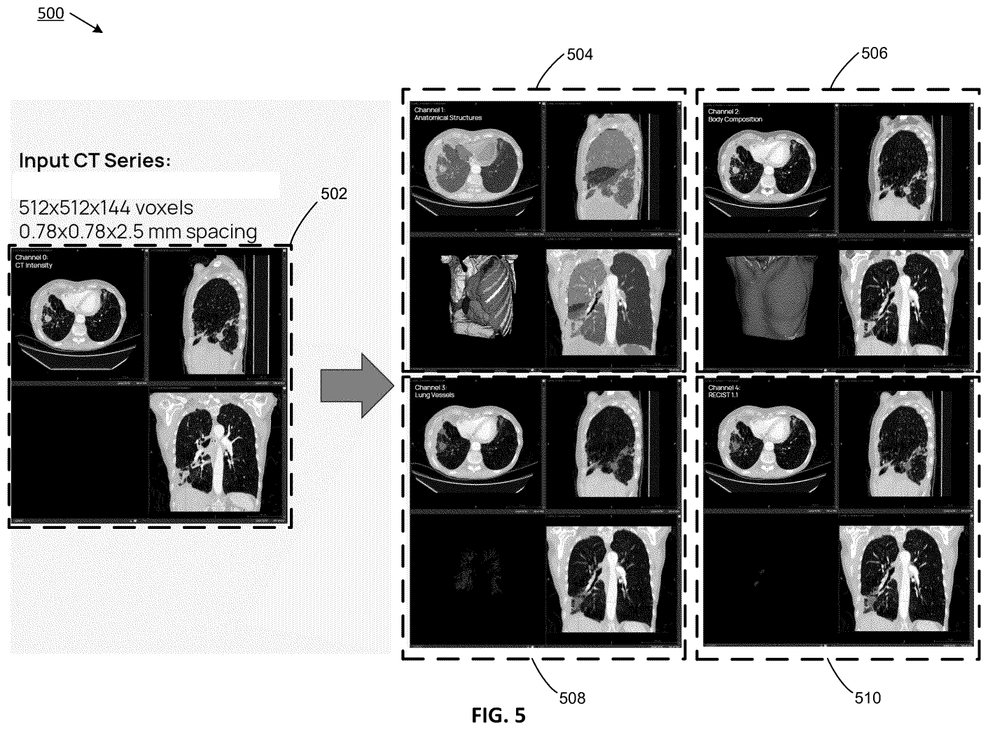

FIG. 5 is a diagram depicting an example environment 500 for generating volumetric segmentation (VS) masks based on a 3D CT scan used to train deep learning models for predicting therapeutic agent responses in specific patients, according to some embodiments. The environment 500 includes an input image 502 of a chest CT scan that was acquired at time of diagnosis for a patient with lung cancer. That is, the CSP agent 130 may select a CT scan that corresponds with a pre-baseline scan 304s or a baseline scan 308s in FIG. 2. The CSP agent 130 of the machine learning architecture 127 identifies, based on the input image 502, the different structures of the patient and generates one or more VS masks (sometimes referred to as channels). A VS mask is a three dimensional (3D) image that can be displayed on a screen and from various views. Each of the VS masks include a plurality of labels (e.g., colors, text, and/or the like) indicating the different structures of the patient.

Specifically, the CSP agent 130 analyzes the input image 502 to identify the anatomical structures (e.g., organs, bones, lobes of lung, etc.) of the patient's body that was captured in the chest CT scan and generates a VS mask 504 that represents the anatomical structures. The VS mask 504 includes a plurality of labels indicating the anatomical structures, such that they can be displayed on the screen from various views.

The CSP agent 130 analyzes the input image 502 to identify the body composition segmentation of the patient's body that was captured in the chest CT scan and generates a VS mask 506 (e.g., channel 2) that represents the body composition segmentation. The VS mask 506 includes a plurality of labels indicating the body composition segmentation, such that they can be displayed on the screen from various views. For example, the CSP agent 130 segments (e.g., splits) the patient's body into three categories: skeletal muscles, subcutaneous fat, and visceral body fat (e.g., hidden fat). Visceral body fat includes, for example, the fat stored deep inside the patient's belly, wrapped around the organs, including the liver and intestines. In some embodiments, the VS mask 506 is representative of the body mass index (BMI) of the patient's body that was captured in the chest CT scan in that the VS mask 506 quantifies the distribution of fat and muscle around the human body.

The CSP agent 130 analyzes the input image 502 to identify the vessel segmentation (e.g., blood vessels) of the patient's body that was captured in the chest CT scan and generates a VS mask 508 (e.g., channel 3) that represents the vessel segmentation of the chest CT scan. The VS mask 508 includes a plurality of labels indicating the vessel segmentation, such that they can be displayed on the screen from various views.

The CSP agent 130 analyzes the input image 502 to identify one or more lesion segmentations (e.g., tumors) of the patient's body that was captured in the chest CT scan and generates a VS mask 510 (e.g., channel 4) that represents the one or more lesion segmentations of the chest CT scan. The VS mask 510 includes a plurality of labels indicating the one or more lesion segmentations, such that they can be displayed on the screen from various views.

Thus, the CSP agent 130 identifies, based on a single CT image (e.g., input image 502), the different structures of the patient and generates one or more CT scans, each representing a unique VS mask of the CT image. That is, as a result of the segmentation process of the input image 502 in FIG. 5, the CSP agent 130 now has access to five 3D images: the input image 502, VS mask 504, VS mask 506, VS mask 508, VS mask 510; where each VS mask is a CT scan that includes one or more labels to identify structures of the patient's body.

The CSP agent 130 combines the input image 502 and the one or more VS masks to generate a 4D image. The CSP agent 130 then provides the single 4D image to the one or more predictive models 140. Each of the one or more predictive models 140 generate, based on the 4D image, a predicted treatment response score that is indictive of the patient's response to a treatment. Therefore, by providing a 4D image (e.g., a pre-segmented 3D image) to the one or more predictive models instead of only a 3D image (as is the case in conventional systems), the one or more predictive model 140 are able to make more informative decisions when predicting the patient's response to treatment based on CT imaging. Advantageously, the predictions made by the one or more predictive models 140 are more efficient and accurate when derived from the analysis of 4D images instead of 3D images because a portion of the analysis (e.g., a processing load) is shifted from the one or more predictive models and placed onto the CSP agent 130, which is more equipped to perform a segmentation of the 3D image.

Although the CT scan in FIG. 5 is of a specific region (e.g., chest) of the patient, the machine learning architecture 127 is configured to process CT scans of any region (e.g., cranial region, thoracic region, pelvic region, and/or the like) of the patient, including a CT scan of the patient's whole body, and regardless of whether the region does or does not include one or more lesions.

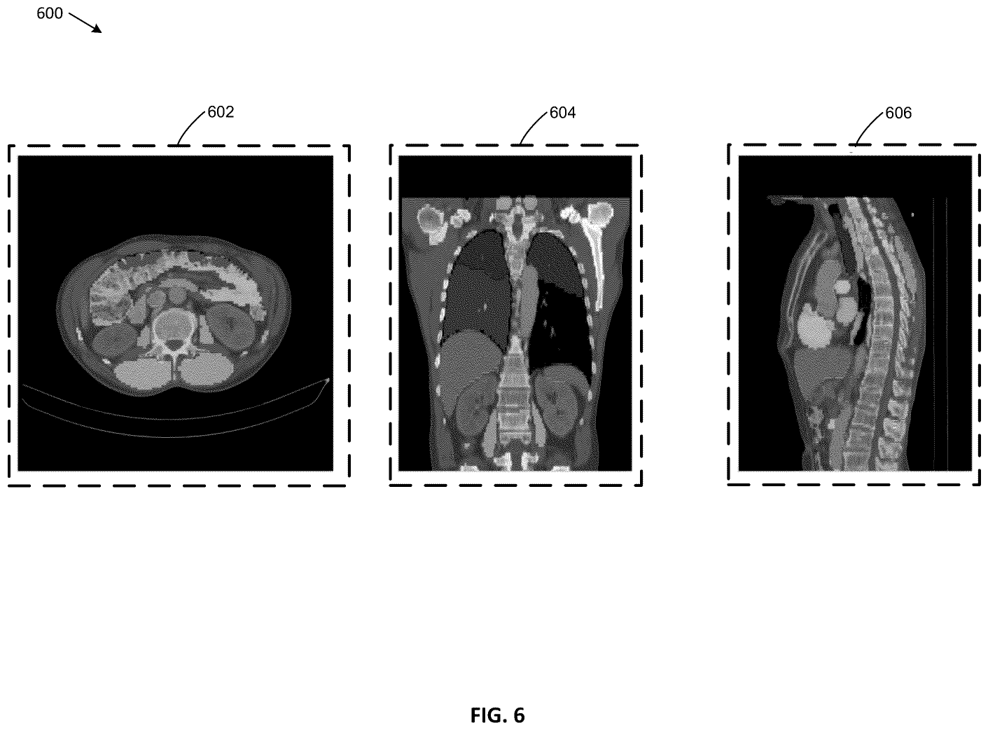

FIG. 6 is a diagram depicting a VS mask that represents the anatomical structures of a patient from an axial view 602, a coronal view 604, and a sagittal view 606, according to some embodiments. The CSP agent 130 may perform an axial slice defined by center of L3 vertebra, or if the CT scan does not include L3 in the field of view, then the CSP agent 130 may use an L2 slice instead. In some embodiments, the CT scan does not include any L3 information of the patient.

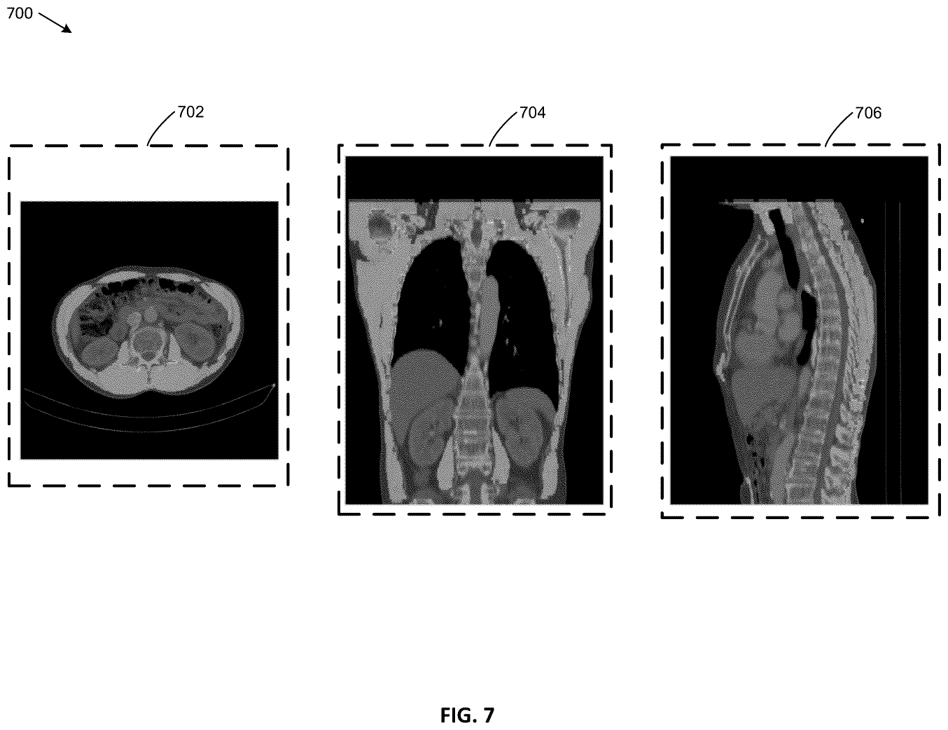

FIG. 7 is a diagram depicting a VS mask that represents the body composition segmentation of a patient using Skeletal Muscle Area (SMA) and Skeletal Muscle Density (SMD) from an axial view 702, a coronal view 704, and a sagittal view 706, according to some embodiments. In some embodiments, the CSP agent 130 may generate a VS mask that also indicates the Skeletal Muscle Index (SMI) of the patient. The CSP agent 130 can calculate SMI by dividing SMA by the patient's height squared, where the units are cm\hat{ }2/m\hat{ }2.

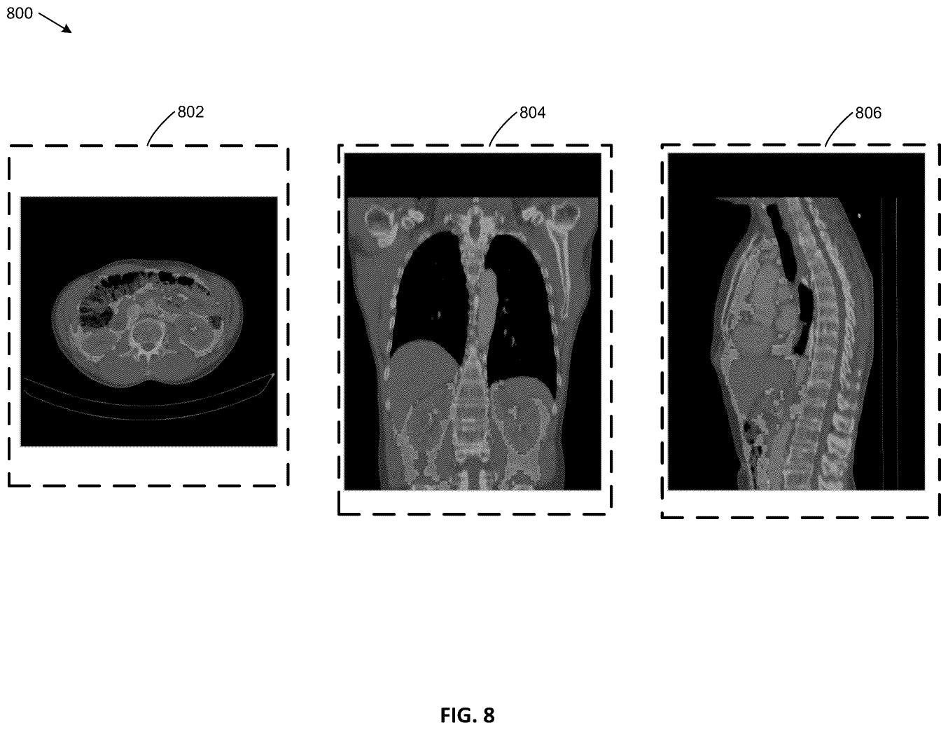

FIG. 8 is a diagram depicting a VS mask that represents the body composition segmentation of a patient using Visceral Fat Area (VFA) and Visceral Fat Density (VMD) from an axial view 802, a coronal view 804, and a sagittal view 806, according to some embodiments.

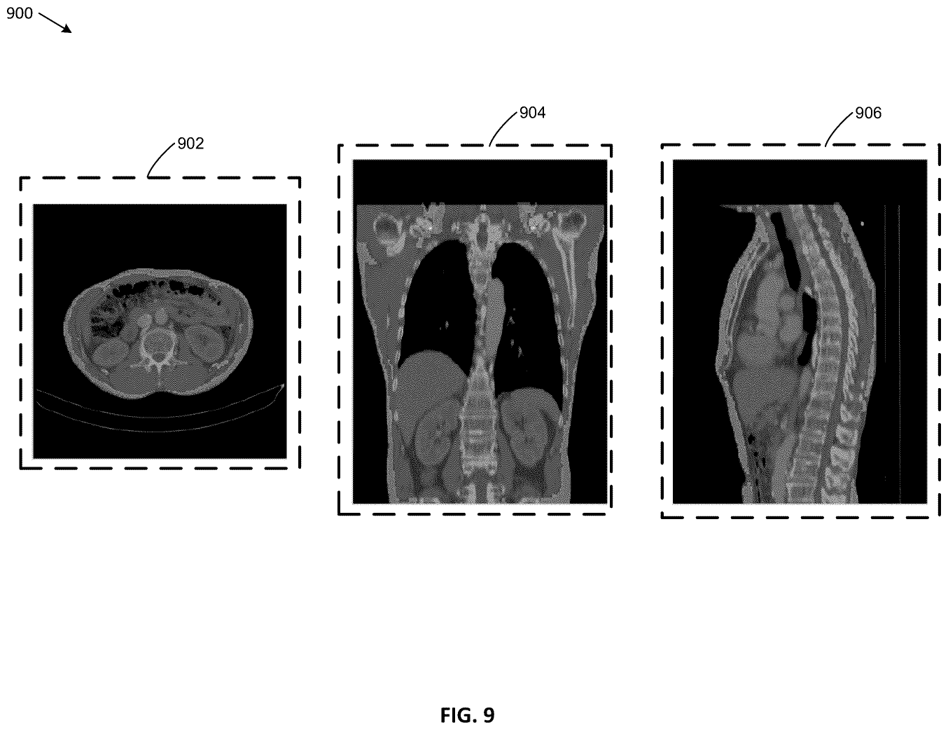

FIG. 9 is a diagram depicting a VS mask that represents the body composition segmentation of a patient using Subcutaneous Fat Area (SFA) and Subcutaneous Fat Density (SFD) from an axial view 902, a coronal view 904, and a sagittal view 906, according to some embodiments.

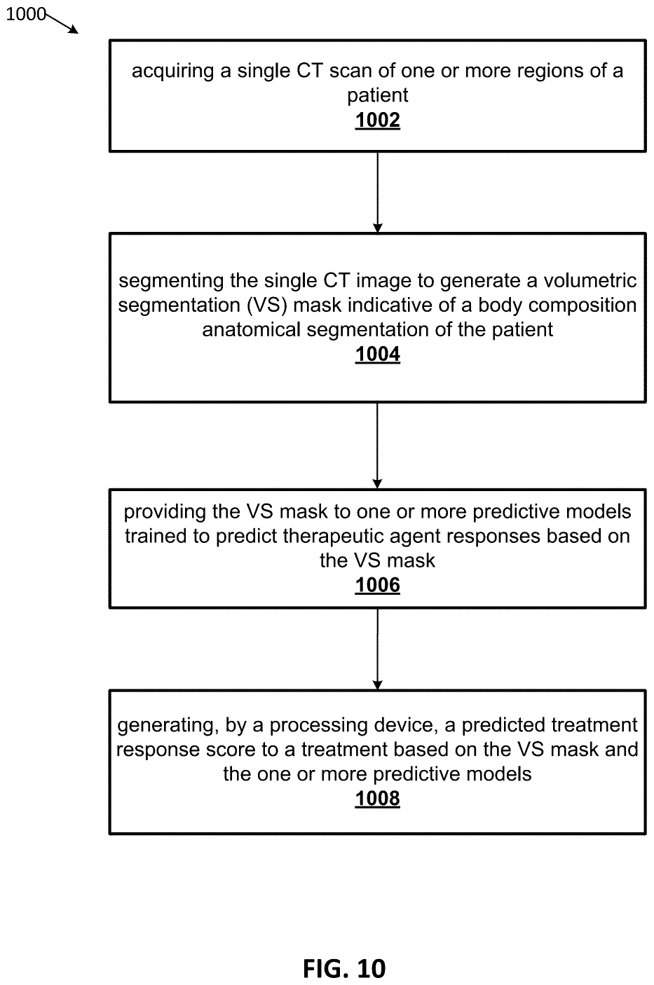

FIG. 10 depicts a flow diagram of a method for segmenting a CT scan into a volumetric segmentation mask indictive of a body composition anatomical segmentation of a patent for predictive modeling of therapeutic agent responses using deep learning analysis, according to some embodiments. Each of the methods described herein (including method 100) may be performed by processing logic that may include hardware (e.g., processing device, circuitry, dedicated logic, programmable logic, microcode, hardware of a device, etc.), software (e.g., instructions run or executed on a processing device), or a combination thereof. In some embodiments, the methods may be performed by processing logic of the machine learning architecture 127 of FIG. 1.

As shown in FIG. 10, the method 1000 includes the block 1002 of acquiring a single CT scan of one or more regions of a patient. The method 1000 includes the block 1004 of segmenting the single CT image to generate a volumetric segmentation (VS) mask indicative of a body composition anatomical segmentation of the patient.

The method 1000 includes the block 1006 of providing the VS mask to one or more predictive models trained to predict therapeutic agent responses based on the VS mask. In some embodiments, the block 1006 may include providing a 4D image that is generated from the VS mask to the deep learning model that is trained, using 4D images, to predict responses to a therapeutic agent based on the 4D image.

The method 1000 includes the block 1008 of generating, by a processing device, a predicted treatment response score to a treatment based on the VS mask and the one or more predictive models. In some embodiments, the block 1008 may include generating, by the processing device, a predicted treatment response score to a treatment based on the 4D image (provided at block 1006) and the one or more predictive models.

In some embodiments, the machine learning architecture 127 in FIG. 1 uses a segmentation algorithm to automatically generate multiple VS masks that describe unique components of the images. The VS masks are then combined with the original CT scan to a create a 4D input to a deep learning model. By training a model using the highly-detailed 4D images (which include labeled information), the machine learning architecture 127 can ensure that the model is accurately trained using the correct information instead of relying on the judgment of the model to blindly identify the correct information from a conventional 3D CT scan (e.g., which does not include labeled information). Thus, providing these image components to the predictive models via segmentation masks facilitates more efficient model training, model computations, and overall quality of the predictive models.

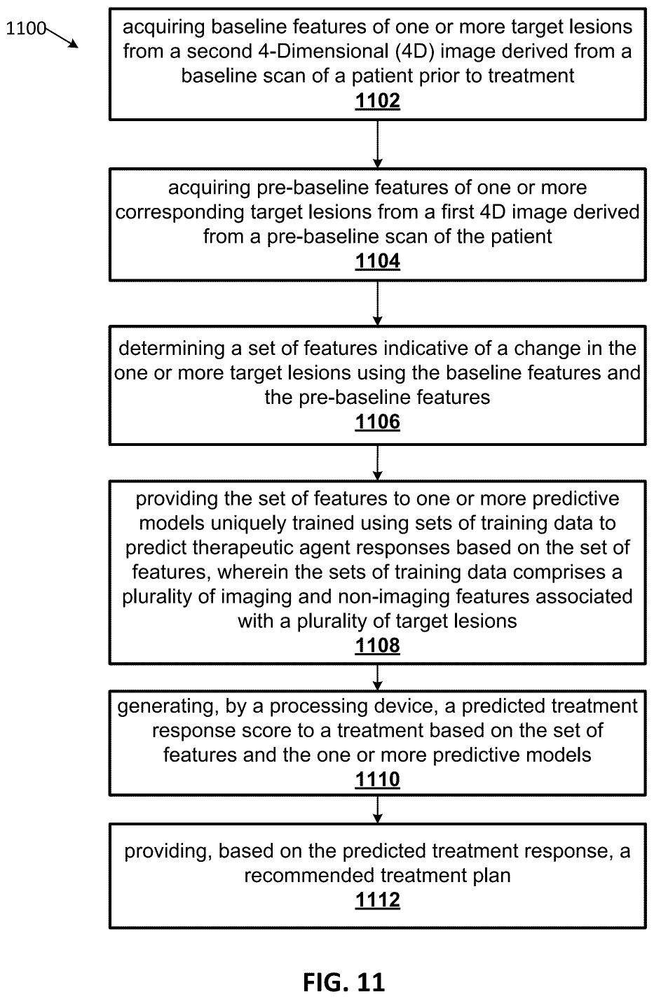

FIG. 11 depicts a flow diagram of a method for predicting therapeutic agent response for a specific patient using deep learning analysis of pre-treatment and intra-treatment serial 4D imaging of that specific patient, according to some embodiments. Each of the methods described herein (including method 1100) may be performed by processing logic that may include hardware (e.g., processing device, circuitry, dedicated logic, programmable logic, microcode, hardware of a device, etc.), software (e.g., instructions run or executed on a processing device), or a combination thereof. In some embodiments, the methods may be performed by processing logic of the machine learning architecture 127 of FIG. 1.

As shown in FIG. 11, the method 1100 includes the block 1102 of acquiring baseline features of one or more target lesions from a 4D image derived from a baseline scan of a patient prior to treatment. In some embodiments, the processing device may acquire the baseline features by retrieving and/or receiving the baseline features from another computing device. In some embodiments, a scan (e.g., pre-baseline scans, baseline scans, follow-up scans) may include one or more treatment images corresponding to 4D images. That is, as discussed with respect to FIG. 10, the CSP agent 130 generates one or more VS masks, and then combines the one or more VS masks and the CT scan to generate a single 4D image. A treatment image (e.g., pre-treatment or post-treatment) may be, but is not limited to, a CT scan, a PET scan, or an MRI scan. In some embodiments, two or more treatment images of a variety of types (e.g., CT scan, PET scan, MRI scan) may be used.

The method 1100 includes the block 1104 of acquiring pre-baseline features of one or more corresponding target lesions from a first 4D image derived from a pre-baseline scan of the patient. In some embodiments, the processing device may acquire the pre-baseline features by retrieving and/or receiving the pre-baseline features from another computing device. In some embodiments, the processing device may acquire the pre-baseline features by identifying one or more corresponding target lesions on the first 4D image.

The method 1100 includes the block 1106 of determining a set of features indicative of a change in the one or more target lesions using the baseline features and the pre-baseline features. In some embodiments, the processing device may determine the set of features by determining baseline features of the one or more target lesions using a second 4D image derived from the baseline scan of the patient, determining pre-baseline features of the one or more target lesions using a first 4D image derived from the pre-baseline scan of the patient, and comparing (e.g., subtracting) the baseline features and the pre-baseline features to determine a difference.

The method 1100 includes the block 1108 of providing the set of features to one or more deep learning models (sometimes referred to as, “predictive models”) uniquely trained using sets of training data to predict therapeutic agent (e.g., immunotherapy treatment) responses based on the set of features (e.g., changes between serial imaging data from different time points). In some embodiments, the sets of training data may include imaging and/or non-imaging features associated with target lesions of a plurality of patients. In some embodiments, the sets of training data may include information indicating one or more changes in lesion volume and/or lesion diameter, and/or other patient-level endpoints including, for example, progression-free survival (PFS), overall survival (OS), clinical benefit, and objective response per RECIST protocol. Examples of a deep learning model include, but are not limited to, artificial neural network, convolutional neural networks, random forest model, support vector machine, and logistic regression model. In another embodiment, a single deep learning model may be used.

In some embodiments, a computing system may train a predictive model to predict a therapeutic agent response using one or more sets of training data, as described herein. In some embodiments, the computing system may train, using one or more sets of training data, a predictive model to predict a therapeutic agent response that is indicative of pseudo-progression based on a change in volume and/or diameter of a lesion of a patient.

The deep learning models may utilize a variety of suitable training methods as discussed herein. For example, in one embodiment, the deep learning models use a population of training subjects and a plurality of images associated with each of a plurality of training subjects as training data. In another embodiment, the deep learning models use calculated subject-specific models as training data. In yet another embodiment, the deep learning models use a combination of the two methods described above. In another embodiment, the models are trained on different data, using different techniques, have different objectives, etc., the results of which may be aggregated in a variety of ways.

In one embodiment, the treatment is a PD-1-based treatment. In another embodiment, the treatment is a PD-L1-based treatment. In yet another embodiment, the treatment is a CTLA-4-based treatment, or any other suitable treatment type (e.g., chemotherapy, pharmaceutical-based therapy, radiotherapy, etc.).

The method 1100 includes the block 1110 of generating, by a processing device, a predicted treatment response score (e.g., on a scale representing least likely to have a positive of negative effect to most likely to have a positive or negative effect) to a treatment based on the set of features and the one or more deep learning models. In one embodiment, processing logic generates the predicted treatment response score based on the single pre-treatment image (e.g., a 4D image) and the two or more deep learning models. For example, in one embodiment, results from the different models may be combined (e.g., averaged, or combined in any other way) to generate a single response score.

In one embodiment, the predicted treatment response score includes a prediction of patient progression on a predefined pharmaceutical product. In another embodiment, the predicted treatment response score indicates a prediction of one or more immune-related adverse events associated with the immunotherapy treatment. In one embodiment, the predicted treatment response score may include a predicted likelihood (e.g., a confidence level) of a specific type of response and/or adverse event occurring. In another embodiment, the response score may also include an indication of pseudo-progression, which is characterized by short-term and temporary increase in tumor volume due to natural swelling and/or inflammation (e.g., in response to treatment), rather than progression of disease. In another embodiment, the response score may indicate the likelihood of hyper-progression, which is a serious condition in which progression of disease is accelerated by administration of therapy. In another embodiment, the response score may include an indication of pseudo-progression, which is characterized by short-term and temporary increase in tumor volume due to natural swelling and/or inflammation (e.g., in response to treatment), rather than progression of a disease. In another embodiment, the response score may be formulated to indicate progression-free or overall patient survival in units of months or years.

The method 1100 may include the block 1112 of providing, based on the predicted treatment response, a recommended treatment plan. For example, based on the predicted treatment response, a recommended treatment plan may include an indication of whether a specific pharmaceutical product should be used, a dosage of such product, a timing associated with administering such a product, etc. In one embodiment, the per-lesion immunotherapy and/or chemotherapy response predictions are used to generate a lesion-specific therapy plan to enhance the therapeutic effect in high-risk lesions by combining ongoing systemic therapy with localized therapy. Localized therapy may be any of the following: stereotactic ablative radiation therapy (SBRT), intensity modulated radiation therapy (IMRT), conformal radiation therapy (CRT), radiosurgery, surgical resection, thermal ablation, cryoablation, or high intensity focused ultrasound (HIFU) therapy. In another embodiment, the recommended treatment plan may be to discontinue one or all therapeutic methods to maximize patient's quality of life.

In one embodiment, processing logic may perform a variety of follow-up operations to increase the accuracy of the prediction and/or recommendation. For example, in one embodiment, processing logic may receive an intra-treatment follow-up image, provide the intra-treatment follow-up image to the machine learning model, and generate an updated predicted treatment response score. Processing logic may then provide, based on the updated predicted treatment response score, an updated recommended treatment plan. In one embodiment, the pre-treatment image and intra-treatment follow-up image each comprise a plurality of imaging-based biomarkers.

In a variety of embodiments, processing logic may perform any number of suitable pre- and post-processing operations that may increase the accuracy, efficiency, and/or compatibility of the machine learning model in the context at hand. For example, with respect to preprocessing, traditional radiomics methods may be susceptible to variations in scanner hardware and imaging protocols. The data preprocessing and data augmentation systems described herein are designed to optimize model generalizability and to minimize model susceptibility to imaging hardware and protocol variations.

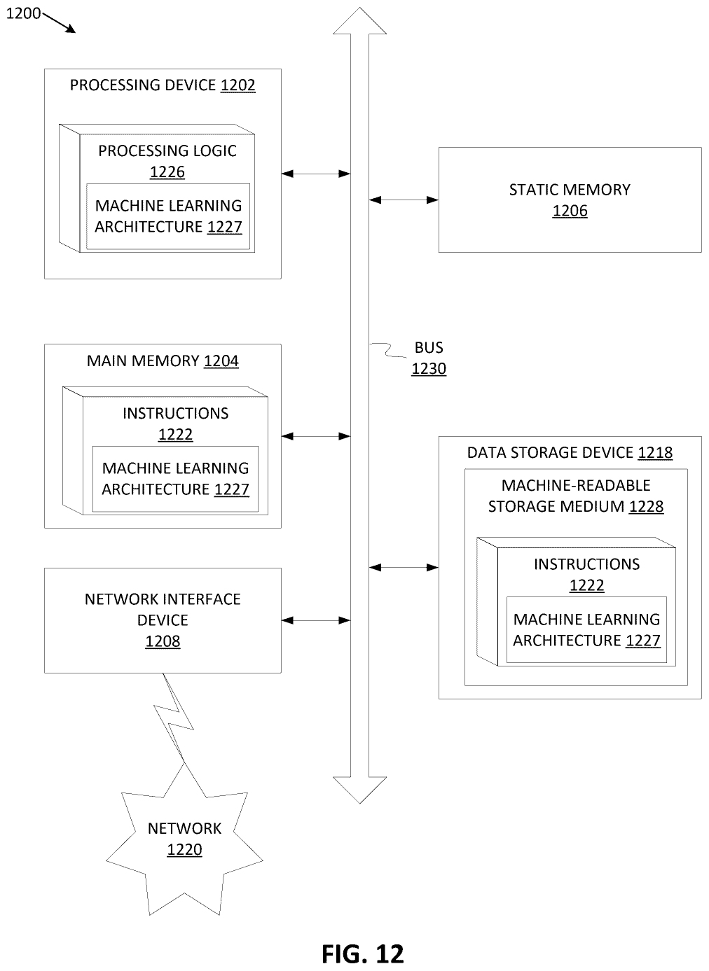

FIG. 12 illustrates a diagrammatic representation of a machine in the example form of a computer system 1200 within which a set of instructions 1222, for causing the machine to perform any one or more of the methodologies discussed herein, may be executed. In alternative embodiments, the machine may be connected (e.g., networked) to other machines in a local area network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a server, a network router, a switch or bridge, a hub, an access point, a network access control device, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. In one embodiment, computer system 1200 may be representative of a server computer system, such as system 100.

The exemplary computer system 1200 includes a processing device 1202, a main memory 1204 (e.g., read-only memory (ROM), flash memory, dynamic random-access memory (DRAM), a static memory 1206 (e.g., flash memory, static random-access memory (SRAM), etc.), and a data storage device 1218, which communicate with each other via a bus 1230. Any of the signals provided over various buses described herein may be time multiplexed with other signals and provided over one or more common buses. Additionally, the interconnection between circuit components or blocks may be shown as buses or as single signal lines. Each of the buses may alternatively be one or more single signal lines and each of the single signal lines may alternatively be buses.

Processing device 1202 represents one or more general-purpose processing devices such as a microprocessor, central processing unit, or the like. More particularly, the processing device may be complex instruction set computing (CISC) microprocessor, reduced instruction set computer (RISC) microprocessor, very long instruction word (VLIW) microprocessor, or processor implementing other instruction sets, or processors implementing a combination of instruction sets. Processing device 1202 may also be one or more special-purpose processing devices such as an application specific integrated circuit (ASIC), a field programmable gate array (FPGA), a digital signal processor (DSP), network processor, or the like. The processing device 1202 is configured to execute processing logic 1226, which may be one example of system 100 shown in FIG. 1, for performing the operations and steps discussed herein.

The data storage device 1218 may include a machine-readable storage medium 1228, on which is stored one or more set of instructions 1222 (e.g., software) embodying any one or more of the methodologies of functions described herein, including instructions to cause the processing device 1202 to execute system 100. The instructions 1222 may also reside, completely or at least partially, within the main memory 1204 or within the processing device 1202 during execution thereof by the computer system 1200; the main memory 1204 and the processing device 1202 also constituting machine-readable storage media. The instructions 1222 may further be transmitted or received over a network 1220 via the network interface device 1208.

The machine-readable storage medium 1228 may also be used to store instructions to perform the methods and operations described herein. While the machine-readable storage medium 1228 is shown in an exemplary embodiment to be a single medium, the term “machine-readable storage medium” should be taken to include a single medium or multiple media (e.g., a centralized or distributed database, or associated caches and servers) that store the one or more sets of instructions. A machine-readable medium includes any mechanism for storing information in a form (e.g., software, processing application) readable by a machine (e.g., a computer). The machine-readable medium may include, but is not limited to, magnetic storage medium (e.g., floppy diskette); optical storage medium (e.g., CD-ROM); magneto-optical storage medium; read-only memory (ROM); random-access memory (RAM); erasable programmable memory (e.g., EPROM and EEPROM); flash memory; or another type of medium suitable for storing electronic instructions.

The preceding description sets forth numerous specific details such as examples of specific systems, components, methods, and so forth, in order to provide a good understanding of several embodiments of the present disclosure. It will be apparent to one skilled in the art, however, that at least some embodiments of the present disclosure may be practiced without these specific details. In other instances, well-known components or methods are not described in detail or are presented in simple block diagram format to avoid unnecessarily obscuring the present disclosure. Thus, the specific details set forth are merely exemplary. Particular embodiments may vary from these exemplary details and still be contemplated to be within the scope of the present disclosure.

Additionally, some embodiments may be practiced in distributed computing environments where the machine-readable medium is stored on and or executed by more than one computer system. In addition, the information transferred between computer systems may either be pulled or pushed across the communication medium connecting the computer systems.

Embodiments of the claimed subject matter include, but are not limited to, various operations described herein. These operations may be performed by hardware components, software, firmware, or a combination thereof.

Although the operations of the methods herein are shown and described in a particular order, the order of the operations of each method may be altered so that certain operations may be performed in an inverse order or so that certain operation may be performed, at least in part, concurrently with other operations. In another embodiment, instructions or sub-operations of distinct operations may be in an intermittent or alternating manner.

The above description of illustrated implementations of the present disclosure, including what is described in the Abstract, is not intended to be exhaustive or to limit the present disclosure to the precise forms disclosed. While specific implementations of, and examples for, the present disclosure are described herein for illustrative purposes, various equivalent modifications are possible within the scope of the present disclosure, as those skilled in the relevant art will recognize. The words “example” or “exemplary” are used herein to mean serving as an example, instance, or illustration. Any aspect or design described herein as “example” or “exemplary” is not necessarily to be construed as preferred or advantageous over other aspects or designs.

Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an implementation” or “one implementation” throughout is not intended to mean the same embodiment or implementation unless described as such. Furthermore, the terms “first,” “second,” “third,” “fourth,” etc. as used herein are meant as labels to distinguish among different elements and may not necessarily have an ordinal meaning according to their numerical designation.

It will be appreciated that variants of the above-disclosed and other features and functions, or alternatives thereof, may be combined into may other different systems or applications. Various presently unforeseen or unanticipated alternatives, modifications, variations, or improvements therein may be subsequently made by those skilled in the art which are also intended to be encompassed by the following claims. The claims may encompass embodiments in hardware, software, or a combination thereof. In the foregoing specification, the disclosure has been described with reference to specific exemplary implementations thereof. It will, however, be evident that various modifications and changes may be made thereto without departing from the broader spirit and scope of the disclosure as set forth in the appended claims. The specification and drawings are, accordingly, to be regarded in an illustrative sense rather than a restrictive sense.

What is claimed, is:

1. A method, comprising:
acquiring a single computer tomography (CT) scan of one or more regions of a patient;
segmenting the single CT scan to generate a volumetric segmentation (VS) mask indicative of a body composition anatomical segmentation of the patient;
providing the VS mask to one or more predictive models trained to predict therapeutic agent responses based on the VS mask; and
generating, by a processing device, a predicted treatment response score to a treatment plan for the patient based on the VS mask and the one or more predictive models.

2. The method of claim 1, further comprising:
training the one or more predictive models using training data that is indicative of a plurality of VS masks associated with a plurality of patients.

3. The method of claim 1, wherein generating the predicted treatment response score is further based on non-imaging information comprising at least one of:
lab values,
patient demographics, or
molecular tests.

4. The method of claim 1, wherein the predicted treatment response score indicates a probability of at least one of progression free-survival or overall survival of patients.

5. The method of claim 1, wherein the single CT scan is acquired prior to administering the treatment plan to the patient.

6. The method of claim 1, wherein the single CT scan includes an L3 vertebral body of the patient.

7. The method of claim 1, wherein the single CT scan does not include an L3 vertebral body of the patient.

8. A treatment analysis system comprising:
a memory to store a pre-treatment image of a target subject; and
a processing device, operatively coupled to the memory, the processing device to:
acquire a single computer tomography (CT) scan of one or more regions of a patient;
segment the single CT scan to generate a volumetric segmentation (VS) mask indicative of a body composition anatomical segmentation of the patient;
provide the VS mask to one or more predictive models trained to predict therapeutic agent responses based on the VS mask; and
generate a predicted treatment response score to a treatment plan for the patient based on the VS mask and the one or more predictive models.

9. The treatment analysis system of claim 8, wherein the processing device is further to:
train the one or more predictive models using training data that is indicative of a plurality of VS masks associated with a plurality of patients.

10. The treatment analysis system of claim 8, wherein to generate the predicted treatment response score is further based on non-imaging information comprising at least one of:
lab values,
patient demographics, or
molecular tests.

11. The treatment analysis system of claim 8, wherein the predicted treatment response score indicates a probability of at least one of progression free-survival or overall survival of patients.

12. The treatment analysis system of claim 8, wherein the single CT scan is acquired prior to administering the treatment plan to the patient.

13. The treatment analysis system of claim 8, wherein the single CT scan includes an L3 vertebral body of the patient.

14. The treatment analysis system of claim 8, wherein the single CT scan does not include an L3 vertebral body of the patient.

15. A non-transitory computer-readable storage medium comprising instructions, which when executed by a processing device, cause the processing device to:
acquire a single computer tomography (CT) scan of one or more regions of a patient;
segment the single CT scan to generate a volumetric segmentation (VS) mask indicative of a body composition anatomical segmentation of the patient;
provide the VS mask to one or more predictive models trained to predict therapeutic agent responses based on the VS mask; and
generate, by the processing device, a predicted treatment response score to a treatment plan for the patient based on the VS mask and the one or more predictive models.

16. The non-transitory computer-readable storage medium of claim 15, wherein to generate the predicted treatment response score is further based on non-imaging information comprising at least one of:
lab values,
patient demographics, or
molecular tests.

17. The non-transitory computer-readable storage medium of claim 15, wherein the predicted treatment response score indicates a probability of at least one of progression-free survival or overall survival of patients.

18. The non-transitory computer-readable storage medium of claim 15, wherein the single CT scan is acquired prior to administering the treatment plan to the patient.

19. The non-transitory computer-readable storage medium of claim 15, wherein the single CT scan includes an L3 vertebral body of the patient.

20. The non-transitory computer-readable storage medium of claim 15, wherein the single CT scan does not include an L3 vertebral body of the patient.
